(19) United States
(12) Patent Application Publication
(10) Pub. No.: US 2011/0045522 A1
(43) Pub. Date: Feb. 24, 2011

(54) METHODS FOR DIAGNOSING DIABETES AND DETERMINING EFFECTIVENESS OF TREATMENTS

(76) Inventors: Danchen Gao, Long Grove, IL (US); Yen-Lun Huang, Taoyuan County (TW); Feng-Min Lin, Taipei City (TW); Wei-Shu Lu, Tucheng City (TW)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/860,042

(22) Filed: Aug. 20, 2010

Related U.S. Application Data
(60) Provisional application No. 61/235,423, filed on Aug. 20, 2009.

(51) Int. Cl. G01N 33/68; C12Q 1/02

(52) U.S. Cl. 435/29; 436/86

(57) ABSTRACT

The invention generally relates to novel methods of measuring the effectiveness of a drug for the treatment of diabetes and methods of diagnosing diabetes. In some embodiments of the invention, IL-1Ra is used as a biomarker to measure the effectiveness of a drug for the treatment of diabetes.

### FIELD OF THE INVENTION

This invention generally relates to novel methods of determining the effectiveness of a compound for the treatment of diabetes and methods of diagnosing diabetes.

### BACKGROUND OF THE INVENTION

Diabetes mellitus is a mammalian condition in which the amount of glucose in the blood plasma is abnormally high. In some instances, elevated glucose levels can lead to higher than normal amounts of a particular hemoglobin, HbA1c. This condition can be life-threatening. High glucose levels in the blood plasma (hyperglycemia) can lead to a number of chronic diabetes syndromes, for example, atherosclerosis, microangiopathy, kidney disorders or failure, cardiac disease, diabetic retinopathy, and other ocular disorders, including blindness.

Diabetes mellitus is known to affect at least 10 million Americans, and millions more may unknowingly have the disease. There are two forms of the disease. In the form of this disease known as Type II, non-insulin dependent diabetes (NIDDM) or adult-onset (as opposed to juvenile diabetes or Type I), the pancreas often continues to secrete normal amounts of insulin. However, this insulin is ineffective in preventing the symptoms of diabetes which include cardiovascular risk factors such as hyperglycemia, impaired carbohydrate (particularly glucose) metabolism, glycosuria, decreased insulin sensitivity, centralized obesity hypertriglyceridemia, low HDL levels, elevated blood pressure and various cardiovascular effects attending these risk factors. Many of these cardiovascular risk factors are known to precede the onset of diabetes by as much as a decade. These symptoms, if left untreated, often lead to severe complications, including premature atherosclerosis, retinopathy, nephropathy, and neuropathy. Insulin resistance is believed to be a precursor to overt. NIDDM and strategies directed toward ameliorating insulin resistance may provide unique benefits to patients with NIDDM.

Current drugs used for managing type II diabetes and its precursor syndromes, such as insulin resistance, fall within five classes of compounds: biguanides, thiazolidinediones, sulfonylureas, benzoic acid derivatives and α-glucosidase inhibitors. Biguanides, e.g., metformin, are believed to prevent excessive hepatic gluconeogenesis. Thiazolidinediones are believed to act by increasing the rate of peripheral glucose disposal. Sulfonylureas, e.g., tolbutamide and glyburide, benzoic acid derivatives, e.g. repaglinide, and α-glucosidase inhibitors, e.g. acarbose, lower plasma glucose primarily by stimulating insulin secretion.

It may be important to determine whether a compound is effective for the treatment of diabetes and to determine an optimal dose of the compound for the treatment of diabetes. It is also desirable to develop new methods of diagnosing and treating diabetes.

### BRIEF SUMMARY OF THE INVENTION

In one embodiment, the invention provides a method of determining the effectiveness of a compound for the treatment of diabetes comprising the steps of:
a. measuring a level of IL-1Ra in a subject having diabetes or in a cell line of a bodily organ targeted by diabetes to obtain a first value;
b. administering to said subject or said cell line said compound in an amount sufficient to determine the effectiveness of said compound;
c. re-measuring the level of IL-1Ra in said subject or said cell line to obtain a second value; and
d. comparing the first value with the second value,
   wherein a higher second value indicates the effectiveness of said compound.

In one embodiment of the invention, the measurement of the IL-1Ra level is also carried out in a control group to ensure that the IL-1Ra level does not change for reasons other than the administration of the tested compound. For example, one may measure the IL-1Ra level in the control group at the same time when the IL-1Ra level is measured in a subject having diabetes or in a cell line of a bodily organ targeted by diabetes (i.e., the experimental group) prior to the administration of the tested compound. Then, the tested compound is administered to the experimental group but not to the control group. Then, the level of IL-1Ra is re-measured in both the experimental group and the control group. If the level of IL-1Ra has not substantially changed in the control group, then the experiment is likely to be valid.

The term “subject” includes humans and animals.

The amount sufficient to determine the effectiveness of a compound will vary with different compounds. It is generally within a skill in the art to determine this amount for each compound to be tested. For example, for diacerin, the amount sufficient to determine the effectiveness of diacerin for the treatment of diabetes is generally within the range of 5-200 mg, and more preferably, 25-150 mg per day.

In another embodiment, the invention provides a method of determining the effectiveness of a compound for the treatment of diabetes comprising the steps of:
a. measuring a level of an intrinsic antagonist of an interleukin receptor, wherein said interleukin is selected from the group consisting of IL-6, IL-12, and IL-18, in a subject having diabetes or in a cell line of a bodily organ targeted by diabetes to obtain a first value;
b. administering to said subject or said cell line said compound in an amount sufficient to determine the effectiveness of said compound;
c. re-measuring the level of said antagonist in said subject or said cell line to obtain a second value; and
d. comparing the first value with the second value,
   wherein a higher second value indicates the effectiveness of said compound.

In one embodiment of the invention, the measurement of the antagonist level is also carried out in a control group to ensure that the antagonist level does not change for reasons other than the administration of the tested compound. For example, one may measure the antagonist level in the control group at the same time when the antagonist level is measured in a subject having diabetes or in a cell line of a bodily organ targeted by diabetes (i.e., the experimental group) prior to the administration of the tested compound. The experimental group and the control group are substantially identical. Then, the tested compound is administered to the experimental group but not to the control group. Then, the antagonist level is re-measured in both the experimental group and the control group. If the antagonist level has not substantially changed in the control group, then the experiment is likely to be valid.

Diabetes may be type I diabetes or type II diabetes.

The level of IL-1Ra or an intrinsic antagonist of an interleukin receptor, wherein said interleukin is selected from the group consisting of IL-1, IL-6, IL-12, and IL-18, may be measured in any suitable bodily fluids, tissues, organs, cells, and supernatants of cell culture.

In a preferred embodiment, the level of IL-1Ra or an intrinsic antagonist of an interleukin receptor, wherein said interleukin is selected from the group consisting of IL-1, IL-6, IL-12, and IL-18, is measured in human kidney cells and/or pancreas cells.

Preferably, the compound is selected from the group consisting of diacerin, rhein, and pharmaceutically acceptable salts thereof.

In another embodiment, the invention provides a method of determining the effectiveness of a compound for the treatment of diabetes comprising determining whether said compound inhibits IL-1β-induced apoptosis of beta cells, wherein inhibition of IL-1β-induced apoptosis of beta cells indicates effectiveness of the compound.

The compound may inhibit IL-1β-induced apoptosis of beta cells via the JUN NH₂-terminal Kinase pathway.

Preferably, the compound is selected from the group consisting of diacerin, rhein, and pharmaceutically acceptable salts thereof.

In another embodiment, the invention provides a method of diagnosing diabetes comprising the steps of:
a. measuring a level of IL-1Ra in a patient suspected of having diabetes to obtain a first value;
b. administering to said patient a diagnostically effective amount of a compound, wherein said compound increases the level of IL-1Ra in patients having diabetes;
c. re-measuring the level of IL-1Ra in said patient to obtain a second value; and
d. comparing the first value with the second value,
   wherein a higher second value indicates the presence of diabetes.

Preferably, the compound is selected from the group consisting of diacerin, rhein, and pharmaceutically acceptable salts thereof.

The level of IL-1Ra may be measured in any suitable bodily fluids, tissues, organs, cells, and supernatants of cell culture. In a preferred embodiment, the level of IL-1Ra is measured in human kidney cells and/or pancreas cells.

In another embodiment, the invention provides a method of diagnosing diabetes comprising the steps of:
a. measuring a level of an intrinsic antagonist of an interleukin receptor, wherein said interleukin is selected from the group consisting of IL-6, IL-12, and IL-18, in a patient suspected of having diabetes to obtain a first value;
b. administering to said patient a diagnostically effective amount of a compound, wherein said compound increases the level of said antagonist in patients having diabetes;
c. re-measuring the level of said antagonist in said patient to obtain a second value; and
d. comparing the first value with the second value,
   wherein a higher second value indicates the presence of diabetes.

Preferably, the compound is selected from the group consisting of diacerin, rhein, and pharmaceutically acceptable salts thereof.

The level of an intrinsic antagonist of an interleukin receptor, wherein said interleukin is selected from the group consisting of IL-1, IL-6, IL-12, and IL-18, may be measured in any suitable bodily fluids, tissues, organs, cells, and supernatants of cell culture.

In a preferred embodiment, the level of said antagonist is measured in human kidney cells and/or pancreas cells.

### BRIEF DESCRIPTION OF THE DRAWINGS

FIG. 1A demonstrates the Standard Curve in an experiment directed to investigating the role of diacerin in protecting proximal renal tubular cells from injury.

FIG. 1B shows a graph demonstrating the dose-dependent effect of diacerin on the production of IL-1Ra during a 16 hour treatment in human kidney proximal cell line (HK-2) cells.

### DETAILED DESCRIPTION OF THE INVENTION

### Definitions

For purposes of the present invention, the term “treatment” refers to reversing, alleviating, inhibiting, or slowing the progress of the disease, disorder, or condition to which such term applies, or one or more symptoms of such disease, disorder, or condition.

The term “therapeutically effective amount” of the compounds and pharmaceutical compositions of the invention refers to a sufficient amount of the compound and/or composition to treat diabetes, at a reasonable benefit/risk ratio applicable to any medical treatment. It will be understood, however, that the total daily usage of the compounds and/or compositions of the present invention will be decided by the attending physician within the scope of sound medical judgment. The specific effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the composition at levels lower than required to achieve the desired therapeutic effect and to gradually increase the dosage until the desired effect is achieved.

The term “diagnostically effective amount” of the compounds and pharmaceutical compositions of the invention refers to a sufficient amount of the compound and/or composition to diagnose diabetes at a reasonable likelihood of certainty.

The term “diacerin” (4,5-bis(acetyloxy)-9,10-dioxo-2-anthracene carboxylic acid or 1,8-Diacetoxy-3-carboxyanthraquinone) refers to the acetylated derivative of rhein (9,10-dihydro-4,5-dihydroxy-9,10-dioxo-2-anthracenecarboxylic acid). Diacerin has the following structural formula:

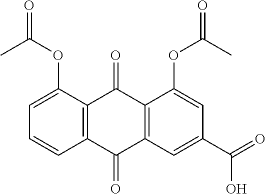

It may also be referred to as 4,5-bis(acetyloxy)-9,10-dihydro-4,5-dihydroxy-9,10-dioxo-2-anthracenecarboxylic acid, 4,5-diacetoxy-9,10-dioxo-9,10-dihydroanthracene-2-carboxylic acid, 4,5-Bis(acetyloxy)-9,10-dihydro-9,10-dioxo-2-anthracenecarboxylic acid, 9,10-dihydro-4,5-dihydroxy-9,10-dioxo-2-anthroic acid diacetate, 1,8-diacetoxy-3-carboxyanthraquinone, diacerhein, and diacetyl rhein.

The term “rhein” is the common name that describes the anthraquinone present in rhubarb (Rhei rhizoma). Rhein possesses the following chemical and non-chemical names: 9,10-dihydro-4,5-dihydroxy-9,10-dioxo-2-anthracenecarboxylic acid; 1,8-dihydroxyanthraquinone-3-carboxylic acid; 4,5-dihydroxyanthraquinone-2-carboxylic acid; chrysazin-3-carboxylic acid; monorhein; rheic acid; cassic acid; arietic acid; and rhubarb yellow.

The term “rhein or diacerin compounds” refers to rhein and diacerin, as well as their analogs, homologues, derivatives, salts, esters, amides and prodrugs. Several patents describe analogs and derivatives of rhein or diacerin that enhance efficacy, increase solubility and bioavailability for human use (U.S. Pat. No. 4,244,968; U.S. Pat. No. 5,986,129; U.S. Pat. No. 5,652,265). The increased solubility and bioavailability has been improved by means of surfactants, water-soluble polymers and micronization techniques (U.S. Pat. No. 6,124,358; European Patent 0809995). Numerous other rhein related derivatives are described in a patent for the use of anthraquinone derivatives for use in arthritis (U.S. Pat. No. 4,346,103). Further, water-soluble formulations for intra-articular injections have also been described (U.S. Pat. No. 4,950,687).

It would be known to a person of skill in the art that other minor modifications to the structure of rhein or diacerin may be made and would result in derivatives of rhein and diacerin that would likely have similar biochemical effects as those disclosed in this invention. Similarly, since the tertiary structures of rhein and diacerin are well known, it would be known to anyone skilled in the art that a structure with a similar tertiary shape as rhein or diacerin (although, chemically different) would be known as an analog of rhein or diacerin and would be predicted to have similar biochemical effects as those disclosed in this invention.

### Methods of Measuring Effectiveness of Compounds to Treat Diabetes

In one embodiment, the invention provides a method of determining the effectiveness of a compound for the treatment of diabetes comprising the steps of:
a. measuring a level of IL-1Ra in a subject having diabetes or in a cell line of a bodily organ targeted by diabetes to obtain a first value;
b. administering to said subject or said cell line said compound in an amount sufficient to determine the effectiveness of said compound;
c. re-measuring the level of IL-1Ra in said subject or said cell line to obtain a second value; and
d. comparing the first value with the second value,
   wherein a higher second value indicates the effectiveness of said compound.

In one embodiment, the measurement of the IL-1Ra level is also carried out in a control group to ensure that the IL-1Ra level does not change for reasons other than the administration of the tested compound. For example, one may measure the IL-1Ra level in the control group at the same time when the IL-1Ra level is measured in a subject having diabetes or in a cell line of a bodily organ targeted by diabetes (i.e., the experimental group) prior to the administration of the tested compound. Then, the tested compound is administered to the experimental group but not to the control group. Then, the level of IL-1Ra is re-measured in both the experimental group and the control group. If the level of IL-1Ra has not substantially changed in the control group, then the experiment is likely to be valid.

In another embodiment, the invention provides a method of determining the effectiveness of a compound for the treatment of diabetes comprising the steps of:
a. measuring a level of an intrinsic inhibitor of an interleukin receptor, wherein said interleukin is selected from the group consisting of IL-6, IL-12, and IL-18, in a subject having diabetes or in a cell line of a bodily organ targeted by diabetes to obtain a first value;
b. administering to said subject or said cell line said compound in an amount sufficient to determine the effectiveness of said compound;
c. re-measuring the level of said inhibitor in said subject or said cell line to obtain a second value; and
d. comparing the first value with the second value,
   wherein a higher second value indicates the effectiveness of said compound.

In one embodiment, the measurement of the antagonist level is also carried out in a control group to ensure that the antagonist level does not change for reasons other than the administration of the tested compound. For example, one may measure the antagonist level in the control group at the same time when the antagonist level is measured in a subject having diabetes or in a cell line of a bodily organ targeted by diabetes (i.e., the experimental group) prior to the administration of the tested compound. The experimental group and the control group are substantially identical. Then, the tested compound is administered to the experimental group but not to the control group. Then, the antagonist level is re-measured in both the experimental group and the control group. If the antagonist level has not substantially changed in the control group, then the experiment is likely to be valid.

The term “subject” includes humans and animals.

The amount sufficient to determine the effectiveness of a compound will vary with different compounds. It is generally within a skill in the art to determine this amount for each compound to be tested. For example, for diacerin, the amount sufficient to determine the effectiveness of diacerin for the treatment of diabetes is generally within the range of 5-200 mg, and more preferably, 25-150 mg per day.

Diabetes may be type I diabetes or type II diabetes.

The level of IL-1Ra or an intrinsic antagonist of an interleukin receptor, wherein said interleukin is selected from the group consisting of IL-6, IL-12, and IL-18, may be measured in any suitable bodily fluids, tissues, organs, cells, and supernatants of cell culture.

In a preferred embodiment, the level of IL-1Ra or an intrinsic antagonist of an interleukin receptor, wherein said interleukin is selected from the group consisting of IL-6, IL-12, and IL-18, is measured in human kidney cells and/or pancreas cells.

Preferably, the compound is selected from the group consisting of diacerin, rhein, and pharmaceutically acceptable salts thereof.

Diacerin is known for its anti-arthritic activity and has been used clinically to treat osteoarthritis, a degenerative disease resulting in articular cartilage loss with aging.

Diacerin is an inhibitor of interleukin-1 (IL-1). Among many publications on diacerin function in osteoarthritis (OA) treatment, the effect of diacerin and its active metabolite, rhein, was evaluated on the production and function of IL-1β, as well as IL-1 receptor antagonist (IL-1Ra) in human OA cartilage and synovial tissue cultures. The results demonstrated that diacerin and rhein significantly inhibited IL-1β production and increased IL-1Ra levels in cartilage culture media.

Thus, one of the anti-inflammatory functions of diacerin appears to be associated with IL-1Ra.

In another embodiment, the invention provides a method of determining the effectiveness of a compound for the treatment of diabetes comprising determining whether said compound inhibits IL-1β-induced apoptosis of beta cells, wherein inhibition of IL-1β-induced apoptosis of beta cells indicates the effectiveness of the compound.

While not wishing to be bound by any particular theory, it is currently believed that the compound may inhibit IL-1β-induced apoptosis of beta cells via the JUN NH₂-terminal Kinase (JNK) pathway.

Preferably, the compound is selected from the group consisting of diacerin, rhein, and pharmaceutically acceptable salts thereof.

### Methods of Diagnosing Diabetes

One of the findings of the present invention is that IL-1Ra, together with compounds that affect the levels of IL-1Ra, may be used as a biomarker to diagnose diabetes.

Thus, in one embodiment, the invention provides a method of diagnosing diabetes comprising the steps of:
a. measuring a level of IL-1Ra in a patient suspected of having diabetes to obtain a first value;
b. administering to said patient a diagnostically effective amount of a compound, wherein said compound increases the level of IL-1Ra in patients having diabetes;
c. re-measuring the level of IL-1Ra in said patient to obtain a second value; and
d. comparing the first value with the second value,
   wherein a higher second value indicates the presence of diabetes.

Preferably, the compound is selected from the group consisting of diacerin, rhein, and pharmaceutically acceptable salts thereof.

The level of IL-1Ra may be measured in any suitable cells, most preferably, in human kidney cells and/or pancreas cells.

In another embodiment, the invention provides a method of diagnosing diabetes comprising the steps of:
a. measuring a level of an intrinsic antagonist of an interleukin receptor, wherein said interleukin is selected from the group consisting of IL-6, IL-12, and IL-18, in a patient suspected of having diabetes to obtain a first value;
b. administering to said patient a diagnostically effective amount of a compound, wherein said compound increases the level of said antagonist in patients having diabetes;
c. re-measuring the level of said antagonist in said patient to obtain a second value; and
d. comparing the first value with the second value,
   wherein a higher second value indicates the presence of diabetes.

Preferably, the compound is selected from the group consisting of diacerin, rhein, and pharmaceutically acceptable salts thereof.

The level of an intrinsic antagonist of an interleukin receptor, wherein said interleukin is selected from the group consisting of IL-1, IL-6, IL-12, and IL-18, may be measured in any suitable bodily fluids, tissues, organs, cells, and supernatants of cell culture. In a preferred embodiment, the level of said antagonist is measured in human kidney cells and/or pancreas cells.

In another embodiment, the invention provides a method of determining the effectiveness of a compound for the treatment of diabetes comprising the steps of:
a. measuring a level of an interleukin, wherein said interleukin is selected from the group consisting of IL-1, IL-6, IL-12, and IL-18, in a subject having diabetes or in a cell line of a bodily organ targeted by diabetes and to obtain a first value;
b. administering to said subject or said cell line said compound in an amount sufficient to determine the effectiveness of said compound;
c. re-measuring the level of said antagonist in said subject or said cell line to obtain a second value; and
d. comparing the first value with the second value, wherein a lower second value indicates the effectiveness of said compound.

In another embodiment, the invention provides a method of determining the effectiveness of a compound for the treatment of diabetes comprising the steps of:
a. measuring a level of an interleukin receptor, wherein said interleukin receptor is selected from the group consisting of IL-1R, IL-6R, IL-12R, and IL-18R, in a subject having diabetes or in a cell line of a bodily organ targeted by diabetes and to obtain a first value;
b. administering to said subject or said cell line said compound in an amount sufficient to determine the effectiveness of said compound;
c. re-measuring the level of said antagonist in said subject or said cell line to obtain a second value; and
d. comparing the first value with the second value,
   wherein a lower second value indicates the effectiveness of said compound.

### Suitable Pharmaceutical Formulations

While the description primarily discusses formulations and methods utilizing diacerin and/or rhein, a person of skill in the art would readily understand that other compounds may be similarly prepared as pharmaceutical formulations and administered to patients.

The active components described for use herein can be included in pharmaceutically suitable compositions, amenable to delivery to a patient by oral, rectal, parenteral (e.g., intravenous, intramuscular, intraarterial, intraperitoneal, and the like), or inhalation routes, osmotic pump, and the like.

For example, the pharmaceutically acceptable salts may be prepared by reacting diacerin and/or rhein with 1 to 10 equivalents of a base such as sodium hydroxide, sodium methoxide, sodium hydride, potassium t-butoxide, calcium hydroxide, magnesium hydroxide and the like, in solvents like ether, tetrahydrofuran, methanol, t-butanol, dioxane, isopropanol, ethanol etc. Mixture of solvents may also be used. Organic bases such as diethanolamine, α-phenylethylamine, benzylamine, piperidine, morpholine, pyridine, hydroxyethylpyrrolidine, hydroxyethylpiperidine, choline, guanidine and the like, ammonium or substituted ammonium salts, aluminum salts. Amino acids such as glycine, alanine, cystine, cysteine, lysine, arginine, phenylalanine etc may be used for the preparation of amino acid salts. Alternatively, acid addition salts, wherever applicable, are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid, salicylic acid, hydroxynaphthoic acid, ascorbic acid, palmitic acid, succinic acid, benzoic acid, benzenesulfonic acid, tartaric acid, oxalic acid and the like in solvents like ethyl acetate, ether, alcohols, acetone, tetrahydrofuran, dioxane etc. Mixture of solvents may also be used.

The stereoisomers of the compounds suitable for the purposes of this invention may be prepared by using reactants in their single enantiomeric form, in the process wherever possible or by conducting the reaction in the presence of reagents or catalysts in their single enantiomeric form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid, and the like, wherever applicable or by using chiral bases such as brucine, cinchona alkaloids, their derivatives and the like.

Prodrugs of diacerin and/or rhein are also contemplated for use in this invention. A prodrug is an active or inactive compound that is modified chemically through in vivo physiological action, such as hydrolysis, metabolism and the like, into a compound useful in this invention following administration of the prodrug to a patient. The suitability and techniques involved in making and using prodrugs are well known by those skilled in the art.

Various polymorphs of diacerin and rhein compounds of this invention may be prepared by crystallization of diacerin and rhein under different conditions. For example, using different commonly used solvents, or their mixtures for recrystallization; crystallizations at different temperatures; various modes of cooling, ranging from very fast to very slow cooling during crystallizations. Heating or melting the compounds followed by cooling gradually or immediately, one can also obtain polymorphs. The presence of polymorphs may be determined by solid probe NMR spectroscopy, IR spectroscopy, differential scanning calorimetry and powder X-ray diffraction or other such techniques.

Pharmaceutically acceptable solvates of diacerin and/or rhein may be prepared by conventional methods such as dissolving diacerin and rhein in solvents such as water, methanol, ethanol, mixture of solvents such as acetone:water, dioxane:water, N,N-dimethylformamide:water, and the like.

The methods of the present invention may also utilize a pharmaceutical composition containing diacerin and/or rhein, their derivatives, analogs, tautomeric forms, stereoisomers, polymorphs, hydrates, metabolites, prodrugs, pharmaceutically acceptable salts, pharmaceutically acceptable solvates in combination with the usual pharmaceutically employed carriers, diluents and the like, useful for the treatment of and/or prophylaxis of disorders in a mammal.

Suitable pharmaceutically acceptable carriers include solid fillers or diluents and sterile aqueous or organic solutions. The active compound will be present in such pharmaceutical compositions in the amounts sufficient to provide the desired dosage in the range as described above.

Pharmaceutical compositions contemplated for use with the methods of the present invention can be used in the form of a solid, a solution, an emulsion, a dispersion, a micelle, a liposome, and the like, wherein the resulting composition contains one or more of the active compounds contemplated for use herein, as active ingredients thereof, in admixture with an organic or inorganic carrier or excipient suitable for nasal, enteral or parenteral applications. The active ingredients may be compounded, for example, with the usual non-toxic, pharmaceutically and physiologically acceptable carriers for tablets, pellets, capsules, troches, lozenges, aqueous or oily suspensions, dispersible powders or granules, suppositories, solutions, emulsions, suspensions, hard or soft capsules, caplets or syrups or elixirs and any other form suitable for use. The carriers that can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents may be used. The active compounds contemplated for use herein are included in the pharmaceutical composition in an amount sufficient to produce the desired effect upon the target process, condition or disease.

The compositions may be prepared by processes known in the art. The amount of the active ingredient in the composition may be less than 70% by weight. Such compositions typically contain from 1 to 25%, preferably 1 to 15% by weight of active compound, the remainder of the composition being pharmaceutically acceptable carriers, diluents, excipients or solvents.

The composition may be administered using any means known in the art, such as orally, nasally, parenterally, topically, transdermally, or rectally. Also within the scope of the invention is release of the active ingredient from a surgical or medical device or implant, such as stents, sutures, catheters, prosthesis and the like. The device may be coated, embedded or impregnated with the compound. The compositions of the present invention may be also formed as a film. The compositions may be prepared by mixing the active ingredients with a suitable non-irritating excipient, such as cocoa butter, synthetic glyceride esters of polyethylene glycols (which are solid at ordinary temperatures, but liquefy and/or dissolve in the rectal cavity to release the active ingredients), and the like.

In addition, sustained release systems, including semi-permeable polymer matrices in the form of shaped articles (e.g., films or microcapsules) can also be used for the administration of the active compound employed herein.

### Injectable Dosage Form

The compounds suitable for the purposes of the invention can be administered parenterally. Parenteral administration, if used, is generally characterized by injection. Injectable formulations can be prepared in conventional forms, either as liquid suspensions or solutions, solid forms suitable for solubilization in liquid prior to injection, or as emulsions, by any method or means known or anticipated in the art. Parenteral administration may also involve a slow release or sustained release system. Administration may also be by continuous infusion or bolus dosing sufficient to maintain therapeutic levels.

For parenteral administration, the compounds can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or alkali or alkaline earth metal salts of the compounds. The injectable solutions prepared in this manner can then be, administered intravenously, intraperitoneally, subcutaneously, or intramuscularly, with intramuscular administration being preferred in humans. Buffers, preservatives, antioxidants, and the like can be incorporated as required.

The precise therapeutically effective amount of rhein or diacerin compound depends on individual differences in age, weight, extent of diabetes, the condition of the patient, and on the compound utilized. Generally, rhein or diacerin compounds should be preferably administered in an amount of at least 0.25 mg/kg per injectable dose, more preferably in an amount up to 5 mg/kg per dose. The effective daily dose may be divided into multiple doses. The total daily dose of the compounds of this invention administered to a human may range from about 0.5 to about 10 mg/kg/day. The amount of rhein and/or diacerin administered to effectively treat diabetes may range from about 1 mg to about 1000 mg daily; more preferably, from about 10 mg to about 500 mg daily, and even more preferably from about 25 mg to about 200 mg daily.

### Oral Dosage Form

For oral administration, the compounds can be combined with a suitable solid or liquid carrier or diluent to form capsules, tablets, powders, syrups, solutions, suspensions and the like. The formulation of tablets and capsules, powders, and other compositions containing a known active ingredient is a skill well known and established in the art. Generally, tablets and capsules for oral use will generally include one or more commonly used carriers such as lactose or corn starch. Other optional components for incorporation into an oral formulation may include preservatives, suspending agents, thickening agents, and the like.

When formulations for oral use are in the form of hard gelatin capsules, the active ingredients may be mixed with an inert solid diluent, for example, calcium carbonate, calcium phosphate, kaolin, or the like. They may also be in the form of soft gelatin capsules wherein the active ingredients are mixed with water or an oil medium, for an example, peanut oil, liquid paraffin, olive oil and the like.

In addition, such compositions may contain one or more agents selected from flavoring agents (such as peppermint, oil of wintergreen or cherry), coloring agents, preserving agents, and the like, in order to provide pharmaceutically elegant and palatable preparations. Tablets containing the active ingredients in admixture with non-toxic pharmaceutically acceptable excipients may also be manufactured by known methods. The excipients used may be, for example, (1) inert diluents, such as calcium carbonate, lactose, calcium phosphate, sodium phosphate, and the like; (2) granulating and disintegrating agents, such as corn starch, potato starch, alginic acid, and the like; (3) binding agents, such as gum tragacanth, corn starch, gelatin, acacia, and the like; and (4) lubricating agents, such as magnesium stearate, stearic acid, talc, and the like. The tablets may be uncoated or they may be coated by known techniques to delay disintegration and absorption in the gastrointestinal tract, thereby providing sustained action over a longer period. For example, a time delay material such as glyceryl monostearate or glyceryl distearate may be employed. The tablets may also be coated by the techniques described in the U.S. Pat. Nos. 4,256,108; 4,160,452; and 4,265,874, incorporated herein by this reference, to form osmotic therapeutic tablets for controlled release.

For purposes of oral administration preferable doses may be in the range of from about 0.1 to about 10 mg/kg/day. If desired, the effective daily dose may be divided into multiple doses for purposes of administration; consequently, single dose compositions may contain such amounts or submultiples thereof to make up the daily dose. “Extended release” formulations may provide the opposite effect, with administration less frequently than once daily and single dose compositions thereof containing multiples of a daily dose.

Release of Composition from Medical Devices

Compounds suitable for the purposes of the invention, in particular, diacerin and/or rhein, may be covalently linked or mixed or encapsulated in microcapsules with either polymeric or non-polymeric formulations which may coat, embed or impregnate or otherwise contact a medical device that is commercially available or is in a research and development phase such as an implant, stent, stent graft, vascular graft, indwelling catheter, sutures, catheter, prosthesis and the like. In other cases, the active compound may contact a medical device such as an implant, stent, stent graft, vascular graft, local balloon delivery, indwelling catheter, sutures, catheter, prosthesis and the like without any formulations. Carriers can be either commercially available or in a research and development phase. Representative examples of carriers include poly(ethylene-vinyl acetate), copolymers of lactic acid and glycolic acid, methacrylate co-polymer, poly(caprolactone), poly(lactic acid), copolymers of poly(lactic acid and caprolactone), gelatin, hyaluronic acid, collagen matrices, cellulose, starch, casein, dextran, polysaccharides, fibrinogen, vitamin B12 and albumin, silicone rubber, acrylic polymers, polyethylene, polypropylene, polyamides, polyurethane, vinyl polymers and poly(ethylene-vinyl acetate) copolymers. Polymeric or non-polymeric carriers may be fashioned in a variety of forms to possess desired release characteristics and/or specific desired properties in response to a specific triggering event such as temperature or pH changes.

The following Examples demonstrate some aspects of the invention. The Examples are not meant to limit the invention in any way.

### EXAMPLES

### Example 1

### Functional Analysis of the Expression of IL-1Ra in Insulin-Producing Rat Beta-Cell Line (rat INS-1E) under the Influence of Diacerin

Diabetes is the result of loss of insulin-producing beta cells of the pancreas. Therefore, investigation of the potential protective role of diacerin in this cell type is essential in understanding the potential therapeutic role of diacerin.

Rat insulinoma-derived INS-1E cells constitute a widely used β-cell surrogate since no such human cells are yet available. However, due to their nonclonal nature, INS-1E cells are heterogeneous and are not stable over extended culture periods. Merglen et al, were the first to isolate clonal INS-1E cells from parental INS-1 in 2004 based on both their insulin content and their secretory responses to glucose. Merglen A, Theander S, Rubi B, Chaffard G, Wollheim B, Maechler P. Glucose sensitivity and metabolism-secretion coupling studied during two-year continuous culture in INS-1E insulinoma cells, Endocrinology, 2004 February; 145(2):667-78. They reported the stable differentiated INS-1E beta-cell phenotype over 116 passages (no. 27-142) representing a 2.2-year continuous follow-up. Thus, INS-1E cells can be safely cultured and used within passages 40-100 with average insulin contents of 2.30+/−0.11 μg/million cells.

Glucose-induced insulin secretion was dose-related and similar to rat islet responses. Secretion saturated with a 6.2-fold increase at 15 mm glucose, showed a 50% effective concentration of 10.4 mm. Secretory responses to amino acids and sulfonylurea were similar to those of islets. Moreover, INS-1E cells retained the amplifying pathway, as judged by glucose-evoked augmentation of insulin release in a depolarized state. Regarding metabolic parameters, INS-1E cells exhibited glucose dose-dependent elevations of NAD(P)H, cytosolic Ca(²⁺), and mitochondrial Ca(²⁺) levels. In contrast, mitochondrial membrane potential, ATP levels, and cell membrane potential were all fully activated by 7.5 mm glucose. Using the perforated patch clamp technique, 7.5 and 15 mm glucose elicited electrical activity to a similar degree. A K(ATP) current was identified in whole cell voltage clamp using diazoxide and tolbutamide. As in native β-cells, tolbutamide induced electrical activity, indicating that the K(ATP)conductance is important in setting the resting potential. Therefore, INS-1E cells represent a stable and valuable β-cell model.

Rat INS-1E cell line may be readily obtained from an investigator. The cell culture procedure is well explained by the publication by Merglen et al. It may be followed as described in their publication as illustrated above.

When the INS-1E cells reach confluency, diacerin in physiologically relevant concentrations of 2.0 μg/ml, 2.5 μg/ml, 3.0 μg/ml, 5.0 μg/ml, 10 μg/ml and 20 μg/ml may be added to culture media and incubate for various time points, such as 0 hour, 0.5 hour, 1 hour, 3 hours, 5 hours, 8 hours, 16 hours, 32 hours, and 72 hours. In addition, one group may be unexposed to diacerin with vehicle as a negative control.

At the end of each diacerin exposure, the culture media may be collected and assayed for IL-1Ra and IL-1 beta concentrations using ELISA assays. Triple samples (n=3) may be assayed. The changes may be determined for statistical significance using statistical analysis software.

If INS-1E cells in culture express significantly higher amount of IL-1Ra when exposed to diacerin compared to un-exposed to diacerin (or exposure at 0 hour), it may be concluded that diacerin has protective effects for INS-1E cells in defense against inflammatory molecule IL-1beta.

A profile of IL-1Ra vs. different diacerin concentrations studied, as well as results at various time points, will be used to determine the maximum and/or the range of protective effects.

Similarly, other compounds may be tested to determine their effectiveness in treating diabetes.

### Example 2

### Functional Analysis of Role of Diacerin in Inhibiting IL-1β-induced Rat INS-1E Cell Apoptosis

It is known that pro-inflammatory cytokine IL-1 beta can induce insulin-producing beta-cell's apoptosis via JUN NH₂-terminal Kinase (JNK) pathway. Investigation of the role of diacerin in inhibiting this apoptotic pathway induced by IL-1 beta is essential to understanding the potential role of diacerin in the treatment of diabetes.

To determine whether diacerin can interfere with the IL-1β-induced apoptosis via JNK pathway, INS-1E cells may be pretreated with exendin-4 at 100 nmole/L concentration. Exendin-4 may be used as a positive control and has been reported to inhibit IL-1β-induced ISN-1E cell apoptosis. Diacerin in physiologically relevant concentrations of 2.0 μg/ml, 2.5 μg/ml, 3.0 μg/ml, 5.0 μg/ml, 10 μg/ml and 20 μg/ml, or the vehicle (a negative control) may be incubated for 8 hours, and thereafter, the cells may be incubated with IL-1β (10 ng/ml) for 16 hours. Apoptosis may be determined by scoring cells displaying pycnotic nuclei visualized with Hoechst 33342 nucleic acid stain (Invitrogen).

If diacerin, in its physiological concentrations of 2.0 μg/ml, 2.5 μg/ml, 3.0 μg/ml, 5.0 μg/ml, 10 μg/ml and 20 μg/ml, can prevent the rat INS-1E cell apoptosis induced by pro-inflammatory cytokine IL-1 beta, it will indicate a protective role of diacerin in the context of diabetes, which is a result of pancreatic β-cell death. A profile of IL-1Ra vs. different diacerin concentrations tested, as well as data collected over various time points, may be used to determine the maximum and/or the range of protective effects.

### Example 3

### Functional Analysis of the Protective Role of Diacerin in Human Kidney Cells

Proximal renal tubular cells are among the first cells injured in the development of diabetic nephropathy. This experiment investigated the role of diacerin in protecting proximal renal tubular cells from injury.

Human kidney proximal cell line is commercially available and may be purchased, for example, from American Type Culture Collection (ATCC) under the symbol HK-2.

The immortalized human kidney proximal tubular cell line (HK-2) was cultured in DMEM/F12 containing 2.50 g/l HEPES, 1.80 g/l sodium bicarbonate, 100 U/ml penicillin, 100 mg/ml streptomycin, and 10% FCS at 37° C. in 5% CO₂. After being digested with 0.25% trypsin, 1×105 cells were grown in 50 ml plastic culture bottles. The cells were cultured overnight in DMEM/F12 5% FCS; then, media were changed to fresh DMEM/F12 5% FCS containing diacerin with various concentrations of 2.0 μg/ml, 2.5 μg/ml, 3.0 μg/ml, 5.0 μg/ml, 10 μg/ml, 20 μg/ml, and negative (5% FCS) controls. Cultures were continued for further time points of 0 hour, 0.5 hour, 1 hour, 3 hours, 5 hours, 8 hours, 16 hours, 32 hours, and 72 hours. Upon reaching each time point, the media were collected. The cell culture method is well explained in Yao, et al, Preventive Effects of Salvianolic Acid on Transforming Growth Factorb 1-Induced Epithelial-to-Mesenchymal Transition of Human Kidney Cells, Biol. Pharm. Bull. 2009, 32(5) 882-886.

The collected media at each time point and concentration were assayed for IL-1Ra concentrations using ELISA assays.

### Results

IL-1Ra production was first observed in HK-2 cells at a concentration of 5 μg/ml diacerin during the 16 hours treatment. Diacerin dose-dependently increased IL-1Ra production during the 16 hours treatment in HK-2 cells.

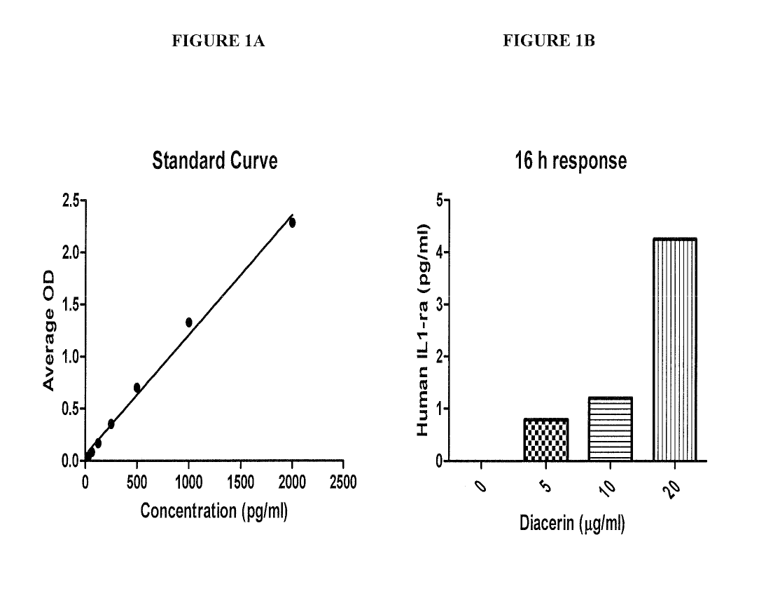

FIG. 1A shows the Standard Curve (i.e., the calibration curve) prepared based on the instructions contained in the ELISA kit using spiked standards. Table 1 shows the data used to construct the Standard Curve.

TABLE 1

| | OD | average OD | concentration (pg/ml) |
|---|---|---|---|
| Blank | 0 | 0 | 0 |
| Standard 1 | 0.044 | 0.042 | 31.2 |
| | 0.04 | | |
| Standard 2 | 0.081 | 0.083 | 62.5 |
| | 0.085 | | |
| Standard 3 | 0.168 | 0.168 | 125 |
| | 0.169 | | |
| Standard 4 | 0.345 | 0.354 | 250 |
| | 0.363 | | |
| Standard 5 | 0.679 | 0.702 | 500 |
| | 0.726 | | |
| Standard 6 | 1.278 | 1.326 | 1000 |
| | 1.374 | | |
| Standard 7 | 2.259 | 2.282 | 2000 |
| | 2.305 | | |

FIG. 1B shows a graph demonstrating the dose-dependent effect of diacerin on production of IL-1Ra during the 16 hours treatment in HK-2 cells. At 0 μg/ml concentration of diacerin, the concentration of IL-1Ra was approximately 0; at 5 μg/ml concentration of diacerin, the concentration of IL-1Ra was approximately 0.8 pg/ml; at 10 μg/ml concentration of diacerin, the concentration of IL-1Ra was approximately 1.3 pg/ml; and at 20 μg/ml concentration of diacerin, the concentration of IL-1Ra was approximately 4.2 pg/ml.

The dose-dependent effect of diacerin on production of IL-1Ra was diminished during the 32 hours and the 72 hours treatment. This may be attributed to a greater variability in the data at prolonged exposure times. The results seem to indicate that the optimal exposure time for diacerin in in vitro HK-2 cultures is about 16 hours.

The observed dose-dependent effect of diacerin indicates a potential protective role of diacerin against pro-inflammatory cytokine IL-1β.

1. A method of determining the effectiveness of a compound for the treatment of diabetes comprising the steps of:
a. measuring a level of IL-1Ra in a subject having diabetes or in a cell line of a bodily organ targeted by diabetes to obtain a first value;
b. administering to said subject or said cell line said compound in an amount sufficient to determine the effectiveness of said compound;
c. re-measuring the level of IL-1Ra in said subject or said cell line to obtain a second value; and
d. comparing the first value with the second value,
wherein a higher second value indicates the effectiveness of said compound.

2. The method of claim 1, further comprising measuring a level of IL-1Ra in a control group.

3. The method of claim 1, wherein the level of IL-1Ra is determined in bodily fluids or tissues.

4. A method of determining an effectiveness of a compound for the treatment of diabetes comprising determining whether said compound inhibits IL-1β-induced apoptosis of beta cells, wherein inhibition of IL-1β-induced apoptosis of beta cells indicates the effectiveness of the compound.

5. A method of determining the effectiveness of a compound for the treatment of diabetes comprising the steps of:
a. measuring a level of an intrinsic antagonist of an interleukin receptor, wherein said interleukin is selected from the group consisting of IL-6, IL-12, and IL-18, in a subject having diabetes or in a cell line of a bodily organ targeted by diabetes and to obtain a first value;
b. administering to said subject or said cell line said compound in an amount sufficient to determine the effectiveness of said compound;
c. re-measuring the level of said antagonist in said subject or said cell line to obtain a second value; and
d. comparing the first value with the second value,
wherein a higher second value indicates the effectiveness of said compound.

6. The method of claim 5, further comprising measuring a level of said antagonist in a control group.

7. The method of claim 1, 4, or 5, wherein the compound is selected from the group consisting of diacerin, rhein, and pharmaceutically acceptable salts thereof.

8. The method of claim 1, 4, or 5 wherein the diabetes is type II diabetes.

9. The method of claim 7, wherein said compound is diacerin.

10. A method of diagnosing diabetes comprising the steps of:
a. measuring a level of IL-1Ra in a patient suspected of having diabetes to obtain a first value;
b. administering to said patient a diagnostically effective amount of a compound, wherein said compound increases the level of IL-1Ra in patients having diabetes;
c. re-measuring the level of IL-1Ra in said patient to obtain a second value, and
d. comparing the first value with the second value,
wherein a higher second value indicates the presence of diabetes.

11. The method of claim 10, wherein the level of IL-1Ra is determined in bodily fluids or tissues.

12. A method of diagnosing diabetes comprising the steps of:
a. measuring a level of an intrinsic antagonist of an interleukin receptor, wherein said interleukin is selected from the group consisting of IL-6, IL-12, and IL-18, in a patient suspected of having diabetes to obtain a first value;
b. administering to said patient a diagnostically effective amount of a compound, wherein said compound increases the level of said antagonist in patients having diabetes;
c. re-measuring the level of said antagonist in said patient to obtain a second value, and
d. comparing the first value with the second value,
wherein a higher second value indicates the presence of diabetes.

13. The method of claim 10 or 12, wherein the compound is selected from the group consisting of diacerin, rhein, and pharmaceutically acceptable salts thereof.

14. The method of claim 10 or 12, wherein the diabetes is type II diabetes.

15. The method of claim 10 or 12, wherein said compound is diacerin.

16. A method of determining the effectiveness of a compound for the treatment of diabetes comprising the steps of:
a. measuring a level of an interleukin, wherein said interleukin is selected from the group consisting of IL-1, IL-6, IL-12, and IL-18, in a subject having diabetes or in a cell line of a bodily organ targeted by diabetes and to obtain a first value;
b. administering to said subject or said cell line said compound in an amount sufficient to determine the effectiveness of said compound;
c. re-measuring the level of said antagonist in said subject or said cell line to obtain a second value; and
d. comparing the first value with the second value,
wherein a lower second value indicates the effectiveness of said compound.

17. A method of determining the effectiveness of a compound for the treatment of diabetes comprising the steps of:
a. measuring a level of an interleukin receptor, wherein said interleukin receptor is selected from the group consisting of IL-1R, IL-6R, IL-12R, and IL-18R, in a subject having diabetes or in a cell line of a bodily organ targeted by diabetes and to obtain a first value;
b. administering to said subject or said cell line said compound in an amount sufficient to determine the effectiveness of said compound;
c. re-measuring the level of said antagonist in said subject or said cell line to obtain a second value; and
d. comparing the first value with the second value;
wherein a lower second value indicates the effectiveness of said compound.
